(19) United States
(12) Patent Application Publication
(10) Pub. No.: US 2013/0096422 A1
(43) Pub. Date: Apr. 18, 2013

(54) INTERVENTIONAL PHOTOACOUSTIC IMAGING SYSTEM

(71) Applicants: Emad Boctor, Baltimore, MD (US); Jin Kang, Ellicott City, MD (US); Stanislav Emelianov, Austin, TX (US); Andrei Karpiouk, Austin, TX (US)

(72) Inventors: Emad Boctor, Baltimore, MD (US); Jin Kang, Ellicott City, MD (US); Stanislav Emelianov, Austin, TX (US); Andrei Karpiouk, Austin, TX (US)

(73) Assignees: THE UNIVERSITY OF TEXAS AT AUSTIN, Austin, TX (US); THE JOHNS HOPKINS UNIVERSITY, Baltimore, MD (US)

(21) Appl. No.: 13/579,113

(22) PCT Filed: Feb. 15, 2011
(86) PCT No.: PCT/US11/24917

§ 371 Date: Dec. 27, 2012

Related U.S. Application Data
(60) Provisional application No. 61/304,626, filed on Feb. 15, 2010.

(51) Int. Cl. A61B 5/00; A61B 18/20; A61B 18/00; A61N 5/10; A61B 10/02

(52) U.S. Cl. CPC A61B 5/0095; A61B 5/6848; A61N 5/1001; A61B 10/0233; A61B 18/00; A61B 5/7225; A61B 5/4381; A61B 5/4331; A61B 5/6852; A61N 5/1007; A61B 18/20
USPC 600/424; 600/407

(57) ABSTRACT

An interventional photoacoustic imaging system and method for cancer treatment comprises an optical source for applying laser energy to optically excite a treatment area, a needle, ablation tool or catheter for inserting the optical source into a body of a patient adjacent the treatment area, and an ultrasonic transducer for detecting the acoustic waves. A processor receives the raw data from the ultrasound system and processes it to thereby form a photoacoustic image of the tissue in real time. As such, image formation may be performed preoperatively, intraoperatively, and postoperatively.

### REFERENCE TO RELATED APPLICATIONS

This application claims the benefit of U.S. Provisional Patent Application No. 61/304,626, filed on Feb. 15, 2010, which is hereby incorporated by reference for all purposes as if fully set forth herein.

### FIELD OF THE INVENTION

This present invention pertains to an interventional photoacoustic imaging system. More particularly the present invention pertains to an interventional photoacoustic imaging system useful for imaging during cancer treatments for the prostate, liver and the like.

### BACKGROUND OF THE INVENTION

Prostate cancer is the leading form of cancer in men in the U.S. For several decades, the definitive treatment for low and medium risk prostate cancer was radical prostatectomy or external beam radiation therapy. More recently, low dose rate (LDR) permanent seed brachytherapy has been used to achieve equivalent outcomes. Brachytherapy is a form of cancer treatment where a radiation source, in the form of radioactive seeds, is positioned at or near the site requiring treatment. For treatment of prostate cancer, brachytherapy seeds are implanted in the tumor and left there to decay over time. Over a period of a few weeks or so, the level of radiation emitted by such seeds will decline to almost zero.

Brachytherapy accounts for a significant and growing proportion of prostate cancer treatments, as it is delivered with minimal invasiveness in an outpatient setting. Brachytherapy is mostly performed with transrectal ultrasound (TRUS) guidance. While TRUS provides adequate imaging of the soft tissue anatomy, it does not allow for localization or precise placement of the implanted brachytherapy seeds, which is a major technical limitation of contemporary brachytherapy. Faulty needle and seed placement often causes insufficient dosing to the cancer site and/or inadvertent radiation of the rectum, urethra, and bladder. The former causes failure of treatment, while the latter results in adverse side effects like rectal ulceration, incontinence, and impotence.

Algorithmic and computational tools are available to optimize a brachytherapy treatment plan intra-operatively. Implant planning is based on idealistic pre-planned seed patterns. These methods, however, critically require that the exact 3-dimensional locations of the implanted seeds are precisely known with respect to the patient's anatomy. Transrectal ultrasound is insufficient for visualizing the seeds within the patient's anatomy.

To provide more accurate imaging, other techniques have emerged. For example, more advanced ultrasound image processing, which incorporate additional modalities like X-rays, have emerged. The use of magnetic resonance imaging has also gained acceptance. However, none of the proposed solutions is effective, simple to use, and low in cost.

Prior to the newer techniques, CT imaging was the only reliable modality to determine if the seeds were implanted at appropriate positions. However, CT imaging is typically acquired weeks after the procedure when edema has subsided. Accordingly, there may cold spots (i.e. under-dosed areas) left in prostate at the end of the procedure. Without having the means of any true quantitative intra-operative dosimetry, these cold spots are left untreated and lead to an increased probability of treatment failure.

In addition to brachytherapy, ablation is also used to treat prostate cancer and the like. In spite of promising results of ablative therapies, significant technical barriers exist with regard to its efficacy, safety, and applicability to many patients. Specifically, these limitations include: (1) localization/targeting of the tumor and (2) monitoring of the ablation zone.

One common feature of current ablative methodology is the necessity for precise placement of the end-effector tip in specific locations, typically within the volumetric center of the tumor, in order to achieve adequate destruction. The tumor and zone of surrounding normal parenchyma can then be ablated. Tumors are identified by preoperative imaging, primarily CT and MR, and then operatively (or laparoscopically) localized by intra-operative ultrasonography (IOUS). When performed percutaneously, trans-abdominal ultrasonography is most commonly used. Current methodology requires visual comparison of preoperative diagnostic imaging with real-time procedural imaging, often requiring subjective comparison of cross-sectional imaging to IOUS. Then, manual free-hand IOUS is employed in conjunction with free-hand positioning of the tissue ablator under ultrasound guidance.

In addition, target motion upon insertion of the ablation probe makes it difficult to localize appropriate placement of the therapy device with simultaneous target imaging. The major limitation of ablative approaches is the lack of accuracy in probe localization within the center of the tumor. In addition, manual guidance often requires multiple passes and repositioning of the ablator tip, further increasing the risk of bleeding and tumor dissemination. In situations when the desired target zone is larger than the single ablation size (e.g. 5-cm tumor and 4-cm ablation device), multiple overlapping spheres are required in order to achieve complete tumor destruction. In such cases, the capacity to accurately plan multiple manual ablations is significantly impaired by the complex 3-dimensional geometrically complex planning required as well as image distortion artifacts from the first ablation, further reducing the targeting confidence and potential efficacy of the therapy. Current monitoring approaches often result in either local failure or in excessively large zones of liver ablation.

Recently, photoacoustic imaging has emerged as a promising new medical imaging modality suitable for various structural, functional, and molecular imaging applications. It is based upon the principle of the photoacoustic effect, simply defined as a physical phenomenon of the conversion of light waves to sound waves depending on a tissue's optical properties. Therefore, photoacoustic imaging involves the detection of sound waves as is also done in ultrasound. In contrast, while ultrasound detects sound waves resulting from scatters or reflections of emitted sound, photoacoustic imaging detects sound waves resulting from the photoacoustic effect generated by a light source (e.g, a laser). However, there has yet been a photoacoustic imaging method and system that provides an integrative and cost effective way to image in real time during cancer treatment, particularly during brachytherapy.

Accordingly, there is a need in the art for a more effective imaging method and system that allows an area under cancer treatment to be imaged in real time.

### SUMMARY

According to a first aspect of the present invention, an interventional photoacoustic imaging system for cancer treatment comprises an energy source including an optical source for applying laser energy to optically excite a treatment area, a device for inserting the optical source into a body of a patient adjacent the treatment area, an ultrasonic transducer for detecting the acoustic waves, and a processor for analyzing the acoustic waves to thereby form a photoacoustic image of the tissue in real time. It is understood that the excitation of sound in a condensed medium is produced by penetrating radiation (beams of protons, electrons, photons, etc.), that is both modulated in intensity and pulsed.

According to a second aspect of the present invention, an interventional photoacoustic imaging method comprises inserting an ultrasonic transducer into a body of a patient, inserting an energy source including an optical source into the body of the patient adjacent a treatment area, illuminating the treatment area with the optical source, detecting acoustic signals generated in the treatment area with the ultrasonic transducer, and analyzing the detected acoustic signals to generate a photoacoustically image of the treatment area.

According to a third aspect of the present invention, a method of imaging implanted brachytherapy seeds comprises implanting a brachytherapy seed into a treatment area. An energy source including an optical source is applied to the treatment area causing the brachytherapy seed to expand and generate acoustic signals. The acoustic signals are detected with an ultrasonic transducer, and are analyzed to generate a photoacoustic image of the seed.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a schematic view of an exemplary interventional photoacoustic imaging system according to the features of the present invention.

FIG. 2A illustrates a partial perspective view of a needle with an optical fiber disposed on the outer surface of the needle according to the features of the present invention.

FIG. 2B illustrates a partial perspective view of a needle with an optical fiber disposed within the needle according to the features of the present invention.

FIG. 3 illustrates a cross sectional view of a catheter including an optical fiber positioned through the urethra of a patient.

FIG. 4 illustrates a schematic showing an experimental setup used to determine photoacoustic properties of tissue and seeds according to the features of the present invention.

FIG. 5A illustrates a schematic showing a basic configuration of the laser flux, transducers and seeds according to features of the present invention.

FIG. 5B illustrates a schematic showing the forward scattering of the acoustic signal towards the transducer according to features of the present invention.

FIG. 6 is a schematic of an experimental setup for acquiring photoacoustic images of brachytherapy seeds embedded in phantom and/or prostate tissue according to features of the present invention.

FIG. 7(a) is a photograph of a one-seed phantom.

FIG. 7(b) is a songraphic image in B-mode of the one-seed phantom.

FIG. 7(c) is a photoacoustic image of the one-seed phantom before beamforming.

FIG. 7(d) is a photoacoustic image of the one-seed phantom after beamforming according to features of the present invention.

FIG. 8(a) is a photograph of a four-seed phantom.

FIG. 8(b) is a songraphic image in B-mode of the four-seed phantom.

FIG. 8(c) is a photoacoustic image of the four-seed phantom before beamforming.

FIG. 8(d) is a photoacoustic image of the four-seed phantom after beamforming according to features of the present invention.

FIG. 9(a) is a photograph of a dog prostate phantom.

FIG. 9(b) is a songraphic image in B-mode of the dog prostate phantom.

FIG. 9(c) is a photoacoustic image of the dog prostate phantom before beamforming.

FIG. 9(d) is a photoacoustic image of the dog prostate phantom after beamforming according to features of the present invention.

FIG. 10 is a schematic view of an exemplary embodiment illustrating the use of photoacoustic imaging in connection with an ablation tool according to features of the present invention.

FIG. 11 is a schematic view of an exemplary embodiment illustrating the use of photoacoustic imaging in connection with an ablation tool according to features of the present invention.

### DETAILED DESCRIPTION PREFERRED EMBODIMENTS

The present invention pertains to an interventional photoacoustic imaging system that may be incorporated into pre-existing medical devices used for treatment of cancer. In particular, a system is developed to allow for photoacoustic imaging of the prostate (or other organ) during cancer treatment, such as hrachytherapy treatment or ablation therapy. In these treatments, precise imaging of the prostate and cancerous tissue, as well as the seeds, is imperative so that effective treatment may be rendered. However, it should be understood that the photoacoustic imaging system and method of the present invention may also be used during laparoscopic surgery, open surgery, and/or natural orifice translumenal surgery (NOTES).

The present invention provides for visualization of the prostate anatomy in real time so that cancer treatments may be rendered effectively in the treatment area of a patient. For example, molecular imaging of the prostate is possible, including edema characterization and mapping radiation dose to actual cancer locations. The present invention also allows for effective imaging of the brachytherapy seeds during the treatment, to thereby verify the proper positioning of the seeds within the prostate according to the treatment plan. Finally, the photoacoustic imaging method and system may be used as an ablation therapy by depositing high laser energy that can cause selective cell necrosis. As used herein, brachytherapy seeds, which are well known, are radioactive sources that are locally deployed adjacent to or implanted within a cancerous site. While brachytherapy seeds are specifically identified, it should be understood that other types of therapeutic seeds may be used in accordance with features of the present invention.

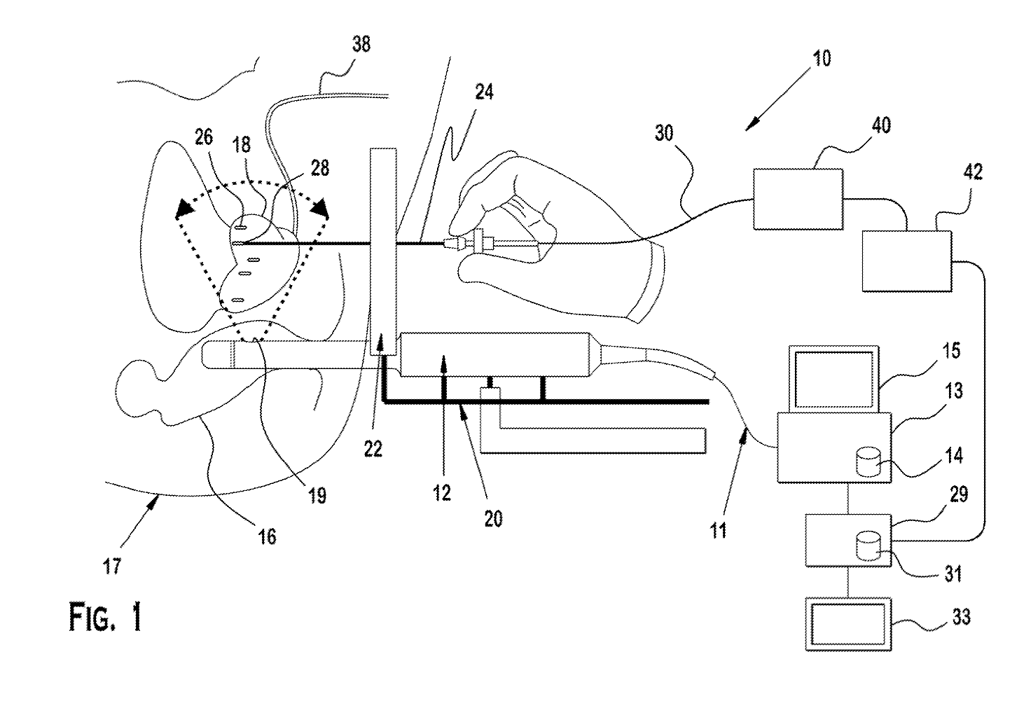

With reference to FIG. 1, an interventional photoacoustic imaging system 10 for imaging of the prostate according to features of the present invention is shown. While system 10 is shown with reference to imaging of the prostate during cancer treatments such as brachytherapy and ablation, it should the system of the present invention may be used in connection with imaging any other organ or area of the body.

As shown in FIG. 1, an interventional photoacoustic imaging system 10 includes an ultrasonic system 11 for generating ultrasound images. The ultrasonic system 11 may include a transrectal probe 12 (TRUS), an ultrasonic processor 13 having a memory device 14, and a user interface 15. According to known principles, the transrectal probe 12 is inserted into a rectum 16 of a patient 17 as to insonate the prostate 18 of the patient 17. Preferably, the transrectal probe 12 includes a scanning aperture 19, through which acoustic signals are transmitted and received. While a transrectal probe has been described in connection with the preferred embodiment, it should be understood that a transvaginal ultrasound may be used for imaging relevant female organs, such as the cervix, ovaries or uterus.

According to known principles, acoustic signals may be acquired by the processor 13, and stored in the memory device 14. The memory device 14 may include one or more computer readable storage media, as well as machine readable instructions for performing processing necessary to form B-mode images. Once the B-mode images are processed, they are displayed on the interface 15, as is known in the art. One skilled in the art will readily appreciate that many variations to ultrasonic system 11 are possible and within the scope of the invention. For example, the ultrasonic system 11 may be a 2-dimensional or 3-dimensional system, but other systems are possible depending on application and design preference.

As is known in the art, a pre-operative treatment plan is generated or the placement of the brachytherapy seeds into the cancerous organ or ablation of the particular area. When performing brachytherapy or ablation therapy, a perineum template may be used to guide the particular needles into the predetermined location. In this regard, a template fixture 20 is used to support a perineum template 22. An example of a perineum template is described in U.S. Pat. No. 6,579,262, the entire disclosure of which is incorporated by reference herein. The perineum template 22 includes a grid of needles holes (not shown) which serve to guide one or more brachytherapy needles or biopsy needles 24 into the treatment area of the patient, for example (and as shown in FIG. 1), the prostate 18, and deploy brachytherapy seeds 26 therein. The template fixture 20 may also be designed to secure the transrectal probe 12 during the procedure.

Typically, B-mode ultrasonic images generated from the transrectal probe 12 are the choice modality for imaging the prostate prior to treatment. However, while ultrasonic images provide adequate imaging of the soil tissue anatomy, they do not allow for localization of the implanted brachytherapy seeds.

With further reference to FIG. 1, the interventional photoacoustic imaging system 10 of the present invention includes an energy source 28 including an optical source to optically excite the prostate 18 tissue and if deployed, the brachytherapy seeds 26. It is understood that excitation of sound in a condensed medium is produced by penetrating radiation (beams of protons, electrons, photons, etc.) that is both modulated intensity and pulsed. According to photoacoustic principles, the prostate tissue 18 and seeds 26 absorb the energy delivered from the energy source, converting it to heat. This leads to transient thermoelastic expansion, which causes acoustic signals to propagate towards the transrectal probe 12. The acoustic signals are received by the transrectal probe 12. However, before the raw data is beamformed, the acoustic signals are streamed to a processor that includes machine readable instructions for analyzing the raw data.

Preferably, a separate computer system 29 is provided to processes the raw data and form photoacoustic images. That is, raw data may be streamed in parallel fashion to the separate processor 29, so that the data may be processed therein to form a photoacoustic image. In this way, the computer system 29 has a memory device 31 including machine readable instructions for performing photoacoustic imaging. Once the photoacoustic images are formed, they may be displayed on an interface 33.

Alternatively, the photoacoustic image may be formed in processor 13 by memory 14. In this way, memory 14 may include additional machine readable instructions to form the photoacoustic image. The photoacoustic images may then be redirected to interface 15, where multiple images may be displayed thereon.

As shown in FIG. 1, the energy source 28 is preferably delivered to the treatment area via the brachytherapy or biopsy needle 24. Due to the small diameter of both the brachytherapy needle and the biopsy needle, the energy source 28 is preferably an optical fiber 30 that optimizes scattering of the light source. One example of an optical fiber that may be used in connection with the present invention is a single mode fiber sold under the product number SM980-5.8-125, manufactured by Fibercore. However, it should be understood that many different kinds of optical fibers may be chosen, depending on application and design preference.

To transmit laser energy to the treatment area, a laser 40 is connected to optical fiber 30. The laser 40, which is controlled by laser controller 42, is preferably a fiber laser capable of generating short pulses of light. The use of fiber lasers and fiber-based components eliminates the need for alignment and makes the whole system rugged, compact, and light. However, any other type of laser system may be incorporated, according to application and design preference.

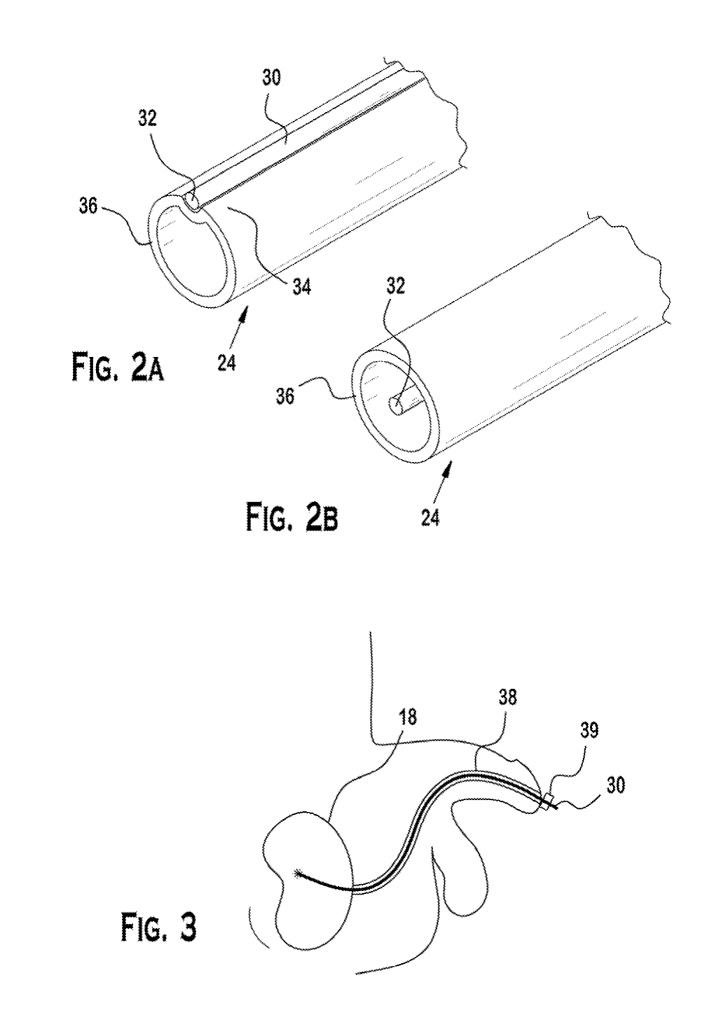

As shown in FIG. 2A, the optical fiber 30 may be placed in a groove 32 on the exterior surface 34 of the hollow shaft 36 of the needle 24. Alternatively, and as shown in FIG. 2B, the optical fiber 30 may be placed within the hollow shaft 36 of the needle 24. While the energy source is shown as being coupled to the needle, it should be understood that the energy source may be deployed in other ways, such as through the urethra 18 or though the rectum 16.

For example, with reference to FIG. 3, an optical fiber 30 may be coupled to catheter 39, such as a Foley catheter. Although the diameter of a Foley catheter is relatively small ranging between about 3-9 mm in diameter, the size is sufficiently large to allow an optical fiber according to features of the present invention to be directed therein. Alternatively, an optical fiber may be manufactured with its own catheter like structure for imaging of the prostate through the urethra.

To image the tissue of the prostate 18, the optical fiber 30 may be advanced at various locations along the urethra 38. That is, optical fiber 30 and accompanying laser source may not be sufficiently strong to generate wavelengths throughout the entire volume of the prostate at a single location. Accordingly, the optical fiber 30 may be positioned at different locations along the urethra 38, so that the entire prostate 18 may be imaged. As such, acquisition of imaging data of the entire prostate may be effected. To facilitate tracking of the catheter 39 in the prostate, sensors, such as electromagnetic sensors, may be attached to the catheter 39, and are tracked by electromagnetic tracking systems (which may be placed under the patient). Photoacoustic phenomenon can also be used to enable tracking of the beam. For example, instead of using 1064 nm wavelength, our system can alternate to 532 nm which is highly absorbed by tissues and will have smaller penetration depth. As such, the system can alternate between 1064 nm wavelength (to image seeds), and 532 nm (to detect the location of the beam).

Preoperatively and postoperatively, insertion of the optical fiber through the urethra 38 may be a preferred method of insertion as a catheter causes lesser trauma than insertion of needles. However, during the interventional operation, where needles are being inserted according to a preoperative plan, the optical fiber coupled to the needle will allow for frequent and instantaneous imaging adjacent the area to be treated. Nevertheless, both methodologies allow for real time imaging of the prostate and to verify that ablation or seed implantation has occurred at their targeted locations, which may eliminate the need for costly and potentially dangerous CT scans.

In addition, thermal imaging may be performed on the prostate 18. In particular, thermal imaging software may be integrated into processor 13 to track temperature elevations within +/−½ degrees C. In particular, the allowed FDA limit for energy flux (30 mJ/cm²) can potentially elevate the temperature of the irradiated area by a half degree depending on the amount of energy, absorption and laser flux profile. Thermal imaging technology can be used to monitor laser energy deposition. Thermal imaging may also be helpful to track the laser source as it moves during the procedure.

For example, thermal imaging may be used as a quality control to monitor the deposited laser energy. For example, with reference to FIG. 1, a temperature map can be generated by processing the photoacoustic signals generated. Photoacoustic images (which are calibrated to reflect temperature maps) may then be used to monitor the deposited laser energy. A more detailed description of thermal imaging is described in U.S. patent application Ser. No. 12/712,019, the entire disclosure of which is incorporated by reference herein.

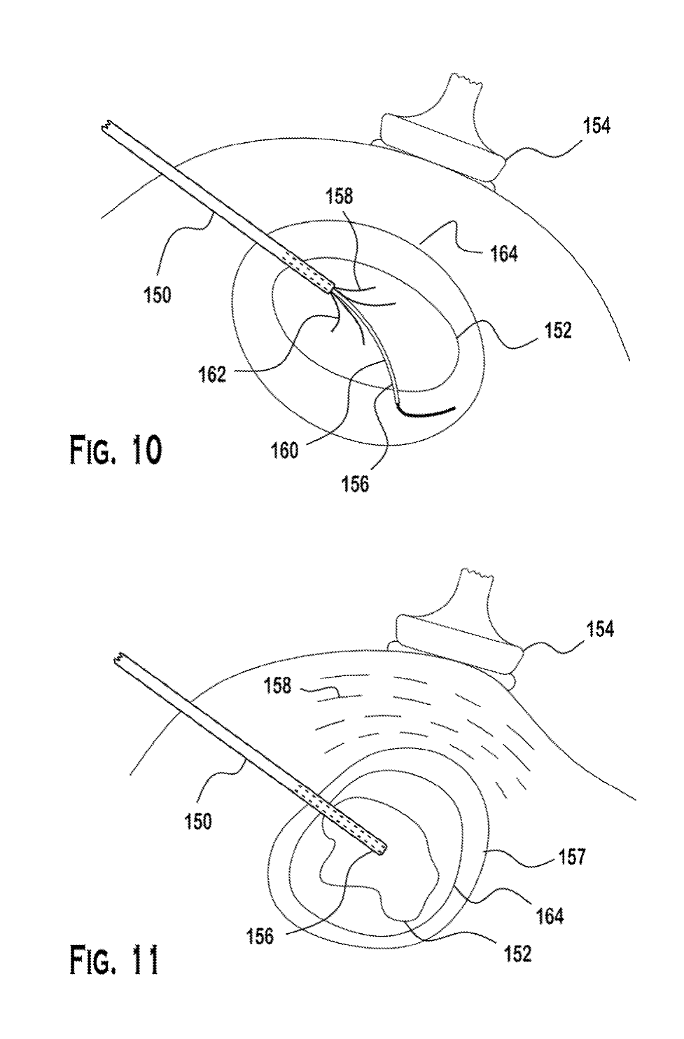

In addition, thermal imaging can be used to monitor ablation therapy. With reference to FIGS. 10 and 11, an ablation system includes an ablation tool 150 for ablating a tumor 152, an ultrasound scanner 154, and an integrated fiber deliver system 156, i.e., laser source. The laser source 156 creates a zone of laser flux 164, which creates photoacoustic signals to be detected by the ultrasound array 154. A control unit (not shown) synchronizes the delivery of both ablation (RF, Microwave, or CW Laser) and pulsed laser energy. Since, the laser source 156 is proximal to the ablation zone 157, the photoacoustic signals will reflect the temperature in this zone and the ultrasound (i.e., photoacoustic) signals 158 can be detected by an ultrasound array 154 located on the organ (as in laparoscopic or open surgery) or the patient skin (as in percutaneous liver ablation).

FIG. 10 shows the use of an active cannula 160 disposed at the end of an ablation tool 150 having tines 162. The structural and functional details of an active cannula is described more thoroughly in U.S. Patent Publication No. 2009/0171271, the entire disclose of which is incorporated by reference herein. The laser source 156, which is incorporated into the active cannula 160 in this embodiment, can be steered within or outside the ablation zone by the active cannular. FIG. 11 shows an alternative embodiment integrating the laser source 156 with the ablation tool 150.

As described above, an optical fiber may be used for ablation itself. In particular, the laser system can be designed to deliver laser energy at a sufficient level for ablation. As such, the optical fiber could serve dual purposes of photoacoustic imaging as well as performing ablation of the cancerous tissue. For example, U.S. Patent Publication No. 2010/0028261, the entire disclosure of which is incorporated by reference herein, relates to molecular specific photoacoustic imaging. By using gold nano particulars to identify cancer markers or contrast agents, the interventional photoacoustic imaging system 10 of the present invention can be tuned (i.e., tune the laser pulse to a specific wavelength, which has maximum absorption by these nano-tubes and minimal interactions with surrounding tissues) to specifically target those gold nano particulars, hence providing molecular imaging. By increasing the laser energy, the high photon counts will cause local ablation only at the region with high absorption (where the tumor markers are located).

Prior to operating the interventional photoacoustic imaging system 10 of the present invention, the optimum laser beam parameters for a particular treatment should be determined. The laser beam parameters will depend upon the optical properties of the treatment area, including the cancerous and normal tissue, as well as any seed that may be implanted therein. The laser beam parameters should be chosen for maximum tissue penetration while minimizing tissue damage. Mainly due to the water and hemoglobin content of tissue, the optical penetration depth of tissue can vary significantly even within tens of nanometers difference in optical wavelength.

In this regard, a laser that has broad wavelength tunability should be used. An example of such a laser is a Ytterbium doped fiber laser (Yb-FL). The Yb-FL laser is capable of generating high energy nanosecond pulses and capable of efficiently fiber optically delivering the beam to the tissue.

In the present invention, it was discovered that when imaging the tissue and edema, the wavelength of the laser energy should be in the range of between about 700 nm and 1350 nm depending on the resolution and the depth of imaging. For the imaging of the metal brachytherapy seeds in the tissue, the wavelength of the laser energy also should be in the range of about 700 nm to 1350 nm. The particular wavelength chosen will depend on the type of imaging that is desired.

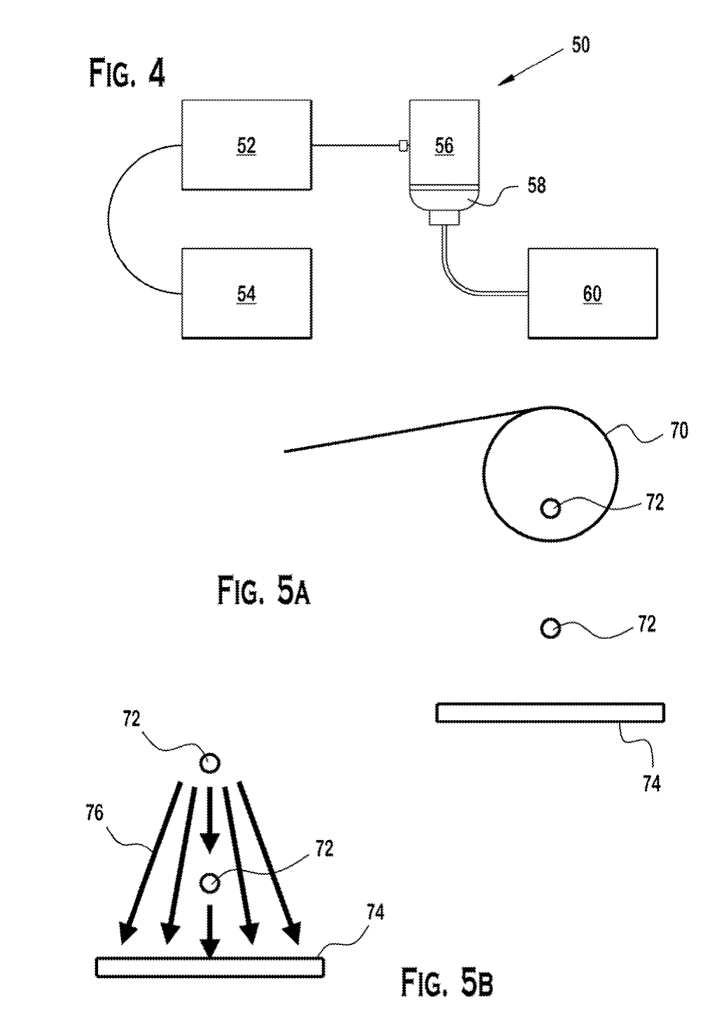

It is also important to understand the photoacoustic properties of the tissue as well as the seeds when designing a system for generating photoacoustic images. With reference to FIG. 4, an experimental system 50 for determining photoacoustic properties of brachytherapy seeds and prostate tissue is shown. The experimental system 50 includes a laser 52 controlled by laser controller 54, which provides pulsed laser energy towards a sample specimen 56. Preferably, the sample specimen 56 is a gel based or ex vivo phantom. As known in the art, a phantom is a non-living model of living tissue, which is used to determine the optical behavior of the tissue.

As shown in FIG. 4, the laser 52 is directed towards the specimen 56, causing the specimen 56 to expand. An ultrasonic transducer 58 is placed adjacent to the specimen 56 to receive acoustic signals therein. Before the data is processed by an ultrasound processor (not shown), the raw data is streamed to a computer processor 60 to thereby create a photoacoustic image. Preferably, the ultrasonic transducer 58 used in the experimental system 50 should be a diagnostic linear array similar to the transducers used in brachytherapy systems (e.g., operating at 7.5 MHz with 60% fractional bandwidth). The experimental system 50 functions to acquire photoacoustic signatures of both the prostate tissue and brachytherapy seeds, which are used as comparative values for generating the photoacoustic image in the interventional photoacoustic imaging system 10 of the present invention (FIG. 1).

### EXAMPLE 1

An experimental system was developed to determine, through photoacoustic imaging, seed location in several test phantoms implanted with brachytherapy seeds. During the experiment, pulsed laser light from a Nd:YAG (neodymium-doped yttrium aluminum garnet) laser was directed towards the phantoms. Due to the intense nature of the generated laser beam, it was necessary to reduce the beam intensity. This was achieved through the use of 2, 45° dielectric mirrors, two black holes (to absorb the laser beam), and an adjustable aperture. The beam was passed through the first 45° dielectric mirror with a significant portion of the beam being deflected into the first black bole. This process was repeated and the resultant beam was passed through an adjustable aperture to further modify beam intensity. As such, the beam intensity could be adjusted to a value of approximately 10 mJ/cm².

The phantoms used were made of two layers having different optical absorption coefficients (similar to the embedded metallic seed in tissue). Specifically, the phantoms were made of polyvinyl chloride-plastisol (PVCP), which mimicked the optoacoustic properties of tissue. The phantom layers were made of PVCP (white) and black plastic color. The optical properties of the phantom could be changed by varying the concentration of PVCP used to prepare it. The white portion was opaque and does not absorb light at 1064 nm. Therefore, the lack of absorption of light at the 1064 nm wavelength allowed the response of the brachytherapy seeds to be observed. Two seeds were implanted into the phantom at a distance of 5 mm apart.

A linear transducer array of 128 elements (used in conjunction with the Ultrasonix open US platform) was positioned adjacent the tissue phantom (using a coupling gel) to create B-mode images and photoacoustic images of two brachytherapy seeds. As shown in FIG. 5A, the laser flux 70 is directed through the phantom containing the brachytherapy seeds 72, and the linear array transducer 74 is disposed opposite the laser flux. In this way, acoustic signals 76 are generated (FIG. 5B), which signals are used for photoacoustic image formation. The recorded acoustic activity is considered a forward scattering signal (as shown in FIG. 5B), which is less attenuated as it travels in one direction, thereby substantially reducing the shadowing effects due to acoustic impedance mismatch.

### EXAMPLE 2

With reference to FIG. 6, a more detailed experimental setup is shown therein, which included a pulsed Nd:YAG laser system 100 (Surelite II, developed by Continuum, Inc. in Santa Clara, Calif.). The laser system 100 was operated at a wavelength of 1064 nm, providing good contrast between the metallic seeds which absorb such light and the soft tissue of the prostate which does not. The Nd:YAG laser 100 operated within an energy density of 40 mJ/cm² (roughly, energy of 40 mJ and a spot size of 1 cm²).

The laser 100 was incorporated into ultrasound system 102. The ultrasound system 102, including transducer 103, was used to detect sound waves generated by the photoacoustic effect. In particular, the ultrasound system 103 was an ultrasonic open research platform known as SONIXCEP manufactured by Ultrasonix Medical Corporation (“Ultrasonic”) located in Richmond, BC, Canada. For faster acquisition, a separately developed data acquisition hardware (DAQ) 104 known as SonixDAQ was connected to the ultrasound system 102 to allow raw pre-beamformed data in parallel to be acquired. That is, as described above, the acquired echos were streamed to a separate processor or processing area before beamforming occurred. In particular, the particular SonixDAQ module 104 used in the experiment supports data acquisition from 128 elements with 12-bit sampling along the external triggering for synchronous data acquisition. In addition, the SonixDAQ has a 16 GB internal memory, a 40 MHz internal clock, and a USB port for transferring data.

To read the raw radio frequency (RF) pre-beamformed data from the SonixDAQ and produce B-mode photoacoustic images, software 106 developed by Ultrasonix (i.e., DAQControl Software) was used. After the raw RF pre-formed data was read from the software 106, a script 108 was implemented to process the RF data into pre-beamformed and standard delay-and-sum B-mode images. Preferably, the software 106 and script 108 are included in the data acquisition hardware (DAQ) 104. However, it should be understood that they may be run in one or more separate processors. In addition, standard ultrasound software 110 is provided for operating the transducer 103, for example, SonixRP Software developed by Ultrasonix. Preferably, the software 110 is run in a processor included the ultrasound system 102.

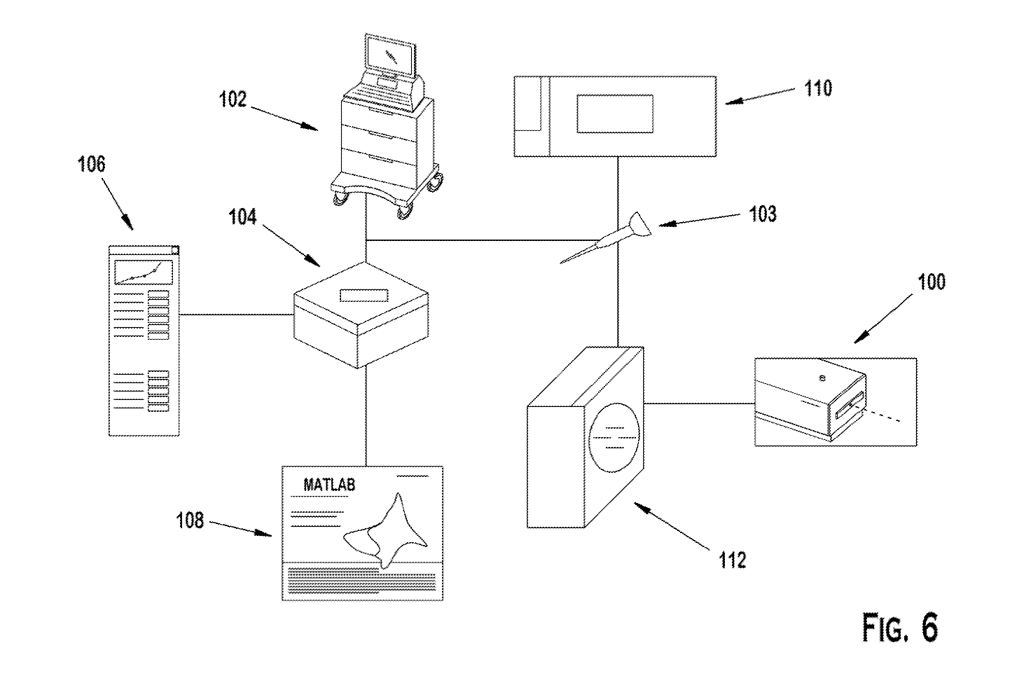

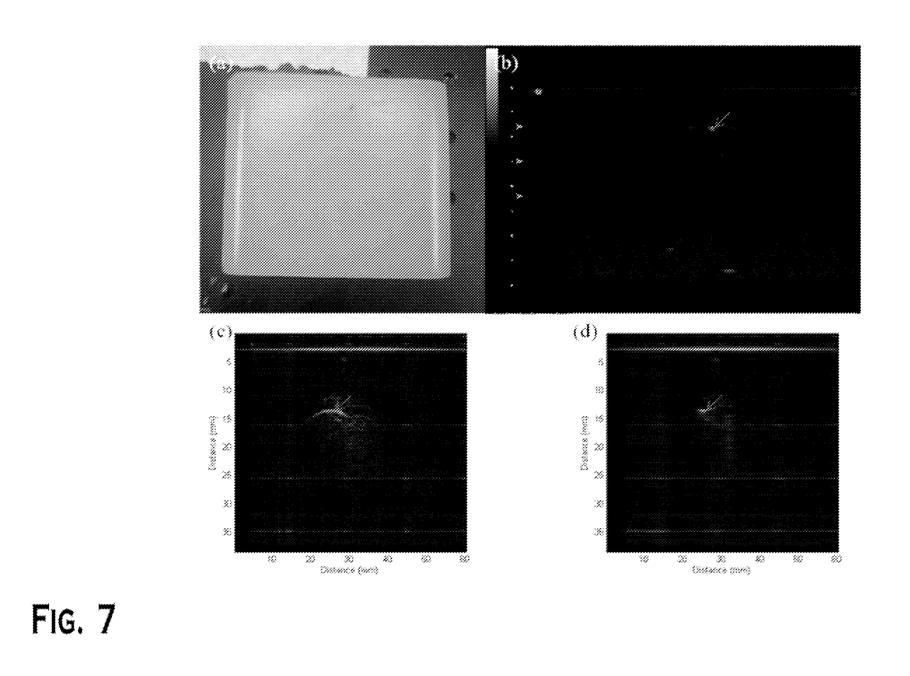

With reference to FIGS. 7-9, three experiments were performed demonstrating the effectiveness of photoacoustic imaging for prostate brachytherapy. Three different phantoms (schematically identified as 112 in FIG. 6) were developed, all using actual decayed palladium-103 brachytherapy seeds encased titanium sold under the name THERASEED and manufactured by Theragenics Corporation, in Buford, Ga.). The first phantom consisted of one seed implanted in gelatin, the second phantom consisted of four seeds implanted in gelatin, while the final phantom consisted of four seeds implanted in an ex vivo dog prostate using gelatin to fix the prostate in place.

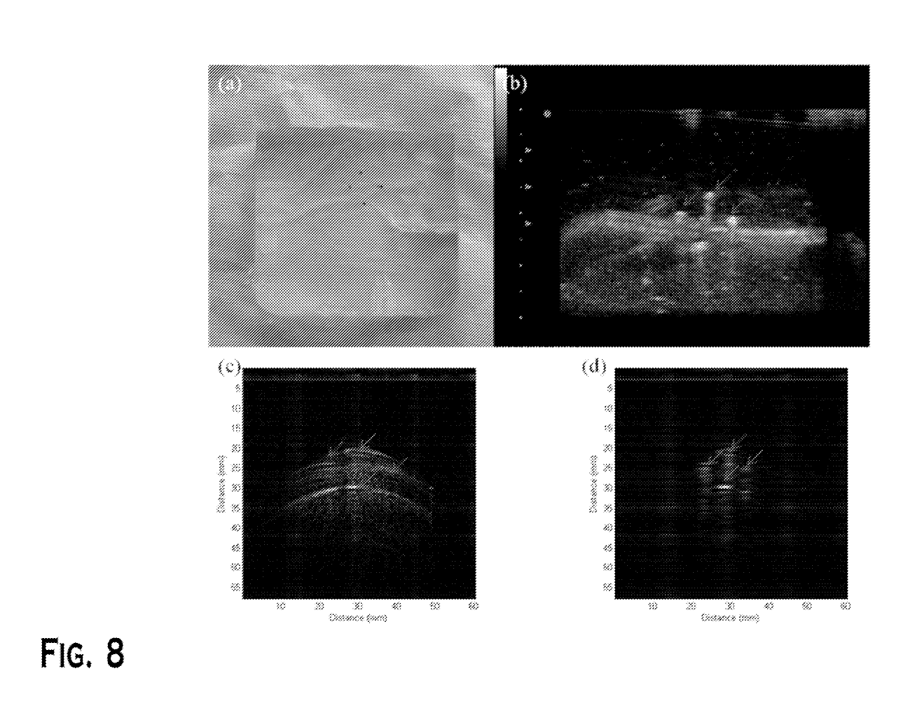

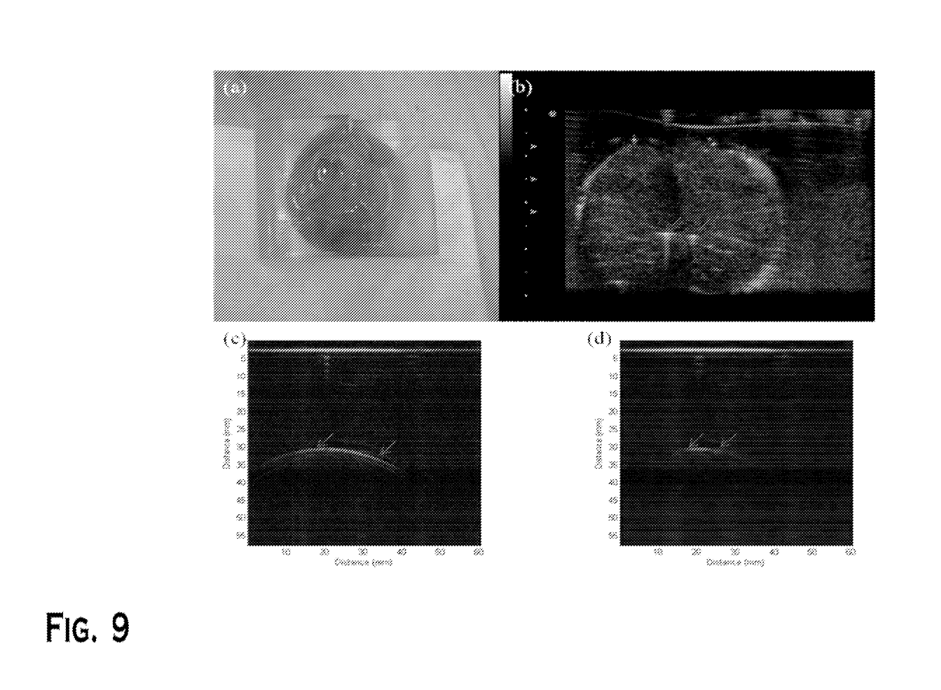

FIG. 7(a) show a photograph of the one-seed phantom, FIG. 7(b) shows the ultrasound image, FIG. 7(c) shows the pre-beamformed photoacoustic image, and FIG. 7(d) shows the delay-and-sum beamformed photoacoustic image. Notably, in FIG. 7(c) the seeds appear as curved streaks before beamforming and in FIG. 7(d), as condensed masses after beamforming. The images from the experimental testing can be improved with more advanced signal processing and hardware improvements. FIGS. 8(a)-(d) and FIGS. 9(a)-(d) show similar results.

To further optimize the photoacoustic image, the ultrasonic system including the beamforming sequence for the linear array may be synchronized with the pulsed laser energy. In this way, laser parameters stored in the laser controller 42 may be acquired by the processor 29 (shown in FIG. 1), and analyzed therein. The synchronization may include numerous transducer array parameters such as effective aperture size, focal depth, frame rate and line density, among others. These parameters affect the resultant location of the photoacoustic seed images due to the delay times generated between the firing sequence of the laser and the time it takes to scan the image plane.

With reference to FIG. 1, operation of the interventional photoacoustic imaging system 10 will be described in more detail. With reference in particular to FIG. 1, a pre-operative imaging of the prostate 18 anatomy may be performed. In particular, the transrectal probe 12 having an aperture 19 is inserted into the rectum 16 of a patient 17 and positioned so that the prostate 18 is in its imaging view. Once the transrectal probe 12 is inserted into the rectum 16, an optical fiber 30 coupled with either a brachytherapy or biopsy needle 24, an ablation tool (FIGS. 10-11), or within a catheter 39 (see FIG. 3), is directed towards the prostate 18.

Once the needle 24 (or ablation tool 150 or catheter 39) is positioned either adjacent and within the prostate 18, the pulsed laser energy is sent through the optical fiber 30, thereby illuminating the neighboring areas. The pulsed laser energy is absorbed by the prostate 18, thereby generating acoustic signals which propagate towards the transrectal probe 12. The signals received in the transrectal probe 12 are acquired by the processor 29 (or processor 13) and analyzed therein. As described with reference to FIG. 3, the optical fiber 30 may be placed at different locations, so that the entire prostate may be imaged. The imaging allows for cancerous tissue to be identified from non-cancerous tissue. Using a variable wavelength source, spectral photoacoustic imaging is possible, including the imaging of the prostate contour, edema and nerve bundle.

Imaging of the seeds 26 is performed similar to imaging of the prostate 18, as described above. In particular, the transrectal probe 12 is positioned in the rectum 16 such that the prostate 18 is in it imaging view. An optical fiber 30 coupled with a needle 24 (or ablation tool 150 as shown in FIGS. 10-11 or catheter 39 as shown in FIG. 3) is displaced through a perineum template 22 (or urethra 38), and directed towards the prostate 18 according to a preoperative plan.

As is known in the art, the needle 24 functions to deliver seeds 26 into predetermined locations. In addition, the needle 24 provides pulsed laser towards the seeds 26, so that embedded seeds 26 may be imaged. Preferably, the pulsed laser energy is about 1064 nm, so that only the seeds are imaged. However, other wavelengths are possible, which would allow both imaging of the prostate 18 with embedded seeds 26. The acoustic signals received in the transrectal probe 12 are acquired by the processor 29 in raw form and analyzed therein, as discussed above.

The present invention integrates photoacoustic imaging into existing ultrasonic systems used for interventional treatments, thereby combining the contrast of optical absorption (prostate tissue vs. metallic seeds) with the spatial resolution of ultrasound in deep regions. Because of strong optical scattering, pure optical imaging in biological tissue has shallow imaging depths. In contrast, pure ultrasonic imaging methods can provide high spatial resolution in deeper regions, primarily because ultrasonic scattering is two to three orders of magnitude weaker than those of optical scattering. Also, current ultrasonic probe array technology allows effective focusing of ultrasound beam through transmit/receive beamforming. Ultrasonic imaging, however, detects mechanical properties derived from acoustic impedance mismatch, which sometimes limit ultrasound imaging quality, sensitivity, and/or depth of penetration.

To this end, photoacoustic imaging overcomes the limitations of existing pure optical and pure ultrasonic imaging. As a result, manifold advantages are obtained, including seed tracking, dynamic dosimetry, effective edema/swelling localization, real-time photoacoustic/ultrasonic fusion, cost effective system, and safety.

Although the present invention has been described in connection with preferred embodiments thereof, it will be appreciated by those skilled in the art that additions, deletions, modifications, and substitutions not specifically described may be made without department from the spirit and scope of the invent on as defined in the appended claims.

1. An interventional photoacoustic imaging system for cancer treatment, comprising:
an energy source including an optical source for applying laser energy to optically excite a treatment area;
means for inserting the optical source into a body of a patient adjacent the treatment area;
an ultrasonic transducer for detecting the acoustic waves; and
a processor for analyzing the acoustic waves to thereby form a photoacoustic image of the tissue in real time.

2. The interventional photoacoustic imaging system of claim 1, wherein said inserting means is a needle and said optical source is an optical fiber coupled within a shaft of the needle, said optical source operatively connected to a pulsed laser source.

3. The interventional photoacoustic imaging system of claim 1, wherein said inserting means is a brachytherapy needle, a biopsy needle or an ablation tool.

4. The interventional photoacoustic imaging system of claim 1, wherein said inserting means is a needle and said optical source is an optical fiber disposed on an outer surface of a shaft of the needle, said optical source operatively connected to a pulsed laser source.

5. The interventional photoacoustic imaging system of claim 4, wherein said inserting means is a brachytherapy needle, a biopsy needle or an ablation tool.

6. The interventional photoacoustic imaging system of claim 5, wherein the laser energy delivered via the ablation tool is adjustable to cause ablation of the tissue.

7. The interventional photoacoustic imaging system of claim 1, wherein said inserting means is a catheter and said optical source is an optical fiber positioned within the catheter, said optical source operatively connected to a pulsed laser source.

8. The interventional photoacoustic imaging system of claim 1, wherein the processor includes a memory encoded with instructions for generating the photoacoustic image.

9. The interventional photoacoustic imaging system of claim 1, wherein the processor is operatively connected to the laser controller for acquiring data related to laser parameters.

10. The interventional photoacoustic imaging system of claim 1, wherein the ultrasonic transducer is transrectal and the treatment area is the prostate.

11. The interventional photoacoustic imaging system of claim 1, wherein the ultrasonic transducer is transvaginal and the treatment area is the cervix.

12. The interventional photoacoustic imaging system of claim 1, wherein the system may be used in laparoscopic surgery, open surgery, or natural orifice translumenal endoscopic surgery for cancer intervention.

13. The interventional photoacoustic imaging system of claim 1, wherein the optical source alternates between an approximately 1064 nm wavelength to image seeds and an approximately 532 nm wavelength to detect the location of the beam.

14. An interventional photoacoustic imaging method, comprising:
inserting an ultrasonic transducer into a body of a patient;
inserting an energy source including an optical source into the body of the patient adjacent a treatment area;
illuminating the treatment area with the optical source; and
detecting acoustic signals generated in the treatment area with the ultrasonic transducer;
analyzing the detected acoustic signals to generate a photo acoustically image of the treatment area.

15. The method of claim 14, wherein the optical source is coupled to a brachytherapy needle, biopsy needle or ablation tool.

16. The method of claim 15, wherein the laser energy delivered via the ablation tool is adjustable to cause ablation of the tissue.

17. The method of claim 14, wherein the optical source is deployed through a catheter positioned in a urethra.

18. The method of claim 14, further comprising:
inserting a brachytherapy needle into the body of the patient;
deploying a brachytherapy seed into the treatment area;
illuminating the treatment area with an energy source including an optical source; and
detecting acoustic signals of the seed in the treatment area with the ultrasonic transducer so as to photoacoustically image the seed.

19. The method of claim 14, wherein the ultrasonic transducer is transrectal and the treatment area is the prostate.

20. The method of claim 14, wherein the method is used in laparoscopic surgery, open surgery, or natural orifice translumenal endoscopic surgery for cancer intervention.

21. The method of claim 14, wherein the optical source alternates between an approximately 1064 nm wavelength to image seeds and an approximately 532 nm wavelength to detect the location of the beam.

22. A method of imaging implanted brachytherapy seeds, comprising:
implanting a brachytherapy seed into a treatment area;
applying an optical source to the treatment area, said optical source causing said brachytherapy seed to expand and generate acoustic signals;
detecting said acoustic signals with an ultrasonic transducer; and
analyzing said acoustic signals to generate a photoacoustic image of said seed.

23. The method of claim 22, wherein said photoacoustic image is generated using delay and sum beamforming.

24. The method of claim 22, further comprising monitoring laser energy deposition through thermal imaging.

25. The method of claim 22, wherein the ultrasound transducer is synchronized with the pulsed laser energy.

26. The method of claim 22, wherein the ultrasonic transducer is transrectal and the treatment area is the prostate.

27. The method of claim 22, wherein the system may be used in laparoscopic surgery, open surgery, or natural orifice translumenal endoscopic surgery for cancer intervention.

28. The method of claim 22, wherein the optical source alternates between an approximately 1064 nm wavelength to image seeds and an approximately 532 nm wavelength to detect the location of the beam.
